(19) United States
(12) Patent Application Publication
(10) Pub. No.: US 2024/0373747 A1
(43) Pub. Date: Nov. 7, 2024

(54) LIGHT-EMITTING MATERIALS AND LIGHT-EMITTING DEVICES

(71) Applicants: Chengdu BOE Optoelectronics Technology Co., Ltd., Chengdu, Sichuan (CN); BOE Technology Group Co., Ltd., Beijing (CN)

(72) Inventors: Meishan XU, Beijing (CN); Yang LIU, Beijing (CN); Hao WANG, Beijing (CN); Yan ZENG, Beijing (CN)

(21) Appl. No.: 18/031,750

(22) PCT Filed: May 19, 2022
(86) PCT No.: PCT/CN2022/093924

§ 371 Date: Apr. 13, 2023

(51) Int. Cl. H10K 85/60; C07D 209/86; C07D 405/14; C07D 413/14; C07D 487/04; C09K 11/06; H10K 50/12

(52) U.S. Cl. CPC H10K 85/654; C07D 209/86; C07D 405/14; C07D 413/14; C07D 487/04; C09K 11/06; H10K 85/657; H10K 85/6572; H10K 85/6574; C09K 2211/1018; H10K 50/12

(57) ABSTRACT

The present disclosure provides a light-emitting material and a light-emitting device. The light-emitting material includes a host material and a doping material, where the host material includes an N-type material and a P-type material, and the N-type material includes a compound with a structural formula shown in Formula 1 or Formula 2. In the present disclosure, a turn-on voltage of the light emitting device can be reduced.

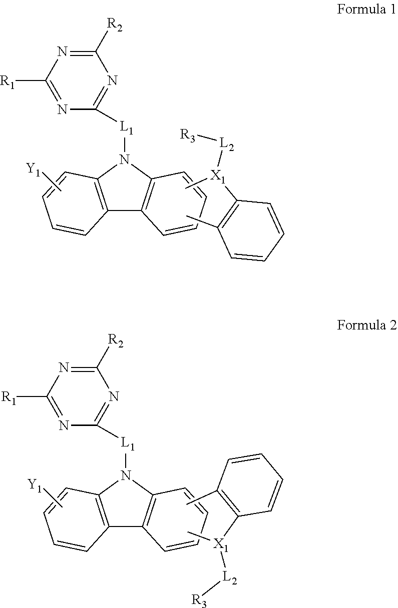

### TECHNICAL FIELD

The present disclosure relates to the field of display technologies, and in particular to a light-emitting material and a light-emitting device.

### BACKGROUND

With the improvement of living standards, OLED light-emitting devices attract more and more attentions. The OLED light-emitting devices have a series of advantages such as full solid-state structure, self-luminescence, fast response, high brightness, full view, flexible display or the like. QLED light-emitting device has advantages such as narrow spectrum of the emitted light, adjustable wavelength of the emitted light or the like. However, a turn-on voltage of current light-emitting devices is relatively high.

### SUMMARY

The object of the present disclosure is to provide a light-emitting material and a light-emitting device, so as to reduce a turn-on voltage of the light-emitting device.

According to an aspect of the present disclosure, there is provided a light-emitting material, the light-emitting material includes a host material and a doping material, where the host material includes an N-type material and a P-type material, and the N-type material includes a compound with a structural formula shown in Formula 1 or Formula 2:

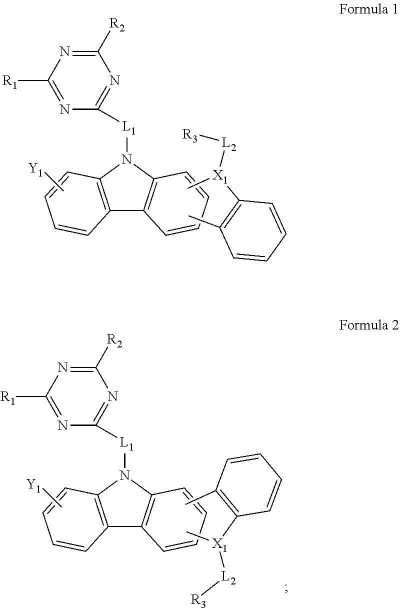

where X₁ is selected from nitrogen and carbon;
Y₁ is selected from hydrogen, deuterium and tritium;
L₁ and L₂ are each independently selected from a single bond and C₆-C₃₀ aryl or heteroaryl;
R₁, R₂ and R₃ are each independently selected from

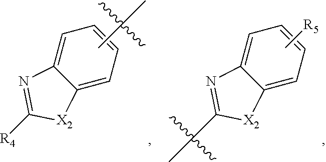

and C₆-C₃₀ aryl or heteroaryl; at least one of R₁, R₂ or R₃ is the

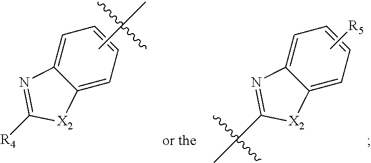

X₂ is selected from sulfur, oxygen and

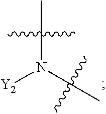

R₄ and R₅ are each independently selected from hydrogen and C₆-C₃₀ aryl or heteroaryl; Y₂ is selected from C₆-C₃₀ aryl or heteroaryl.

In some embodiments, the

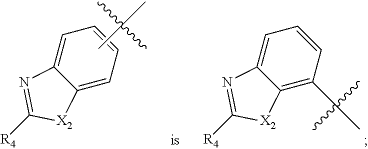

and/or the

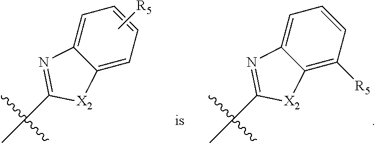

In some embodiments, R₄ and R₅ are each independently selected from hydrogen and phenyl.

In some embodiments, the structural formula shown in Formula 1 is as follows:

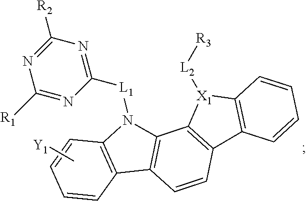

and/or
the structural formula shown in Formula 2 is as follows:

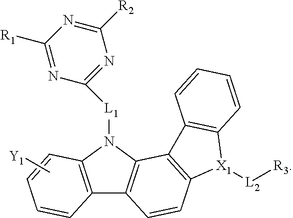

In some embodiments, the structural formula shown in Formula 1 is as follows:

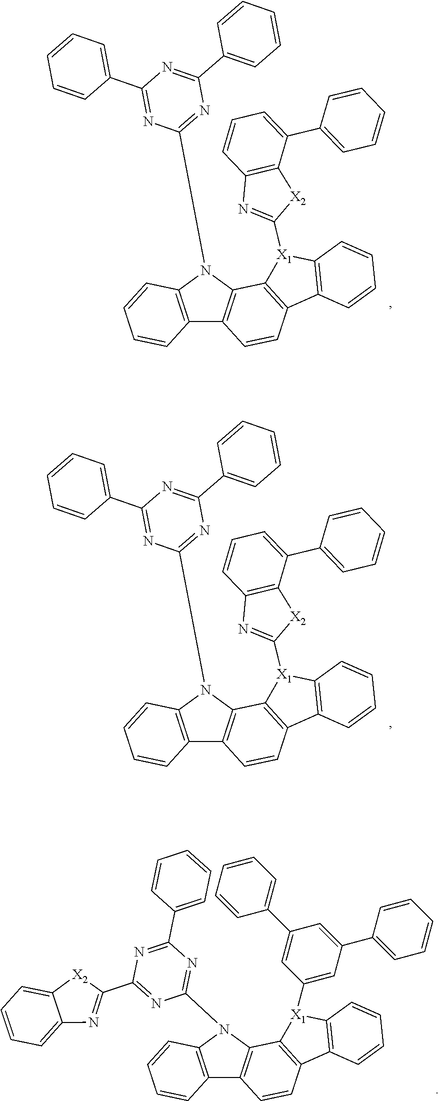

In some embodiments, the doping material is a phosphorescent material.

In some embodiments, the doping material is a green phosphorescent material.

In some embodiments, the P-type material includes a compound with a structural formula shown in Formula 3:

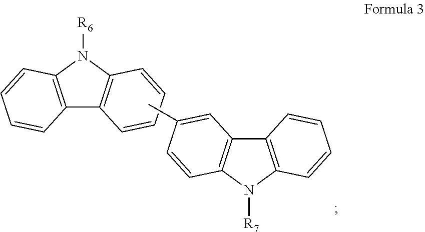

where R₆ and R₇ are each independently C₆-C₃₀ aryl or heteroaryl.

In some embodiments, R₆ and R₇ are each independently selected from structural formulas as follows:

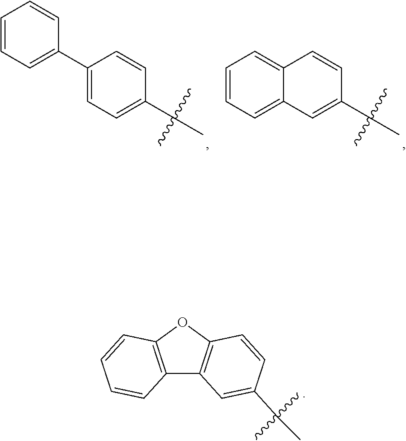 [SMILES: *c1ccc(-c2ccccc2)cc1 ; *c1ccc2ccccc2c1 ; *c1ccc2oc3ccccc3c2c1]

In some embodiments, the structural formula shown in Formula 3 is as follows:

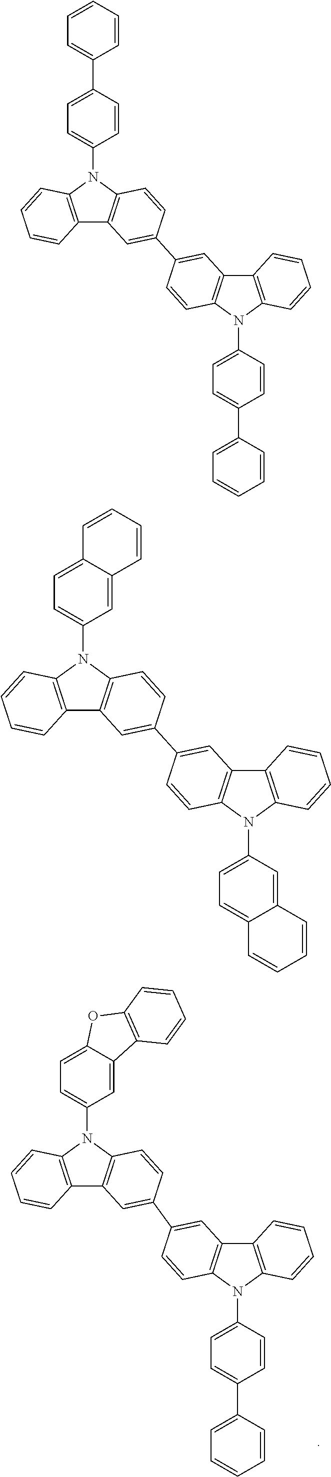 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5ccc6ccccc6c5)ccc43)ccc2c1 ; c1ccc(-c2ccc(-n3c4ccccc4c4cc(-c5ccc6c(c5)c5ccccc5n6-c5ccc6oc7ccccc7c6c5)ccc43)cc2)cc1]

According to an aspect of the present disclosure, there is provided a light-emitting device, including:
an anode and a cathode arranged opposite to each other; and
a light-emitting layer, disposed between the anode and the cathode, where the light-emitting layer includes the light-emitting material.

In some embodiments, the light-emitting device further includes:
an electron transport layer, disposed between the light-emitting layer and the cathode; and
a hole blocking layer, disposed between the electron transport layer and the light-emitting layer, where the hole blocking layer includes a compound with a structural formula shown in Formula 4:

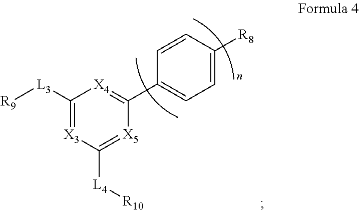

where n is a positive integer greater than or equal to 1 and less than or equal to 5;
X₃, X₄ and X₅ are each independently selected from carbon and nitrogen;
L₃ and L₄ are each independently selected from a single bond and C₆-C₃₀ aryl or heteroaryl;
R₈ is selected from hydrogen, C₆-C₃₀ aryl or heteroaryl;
R₉ and R₁₀ are each independently selected from

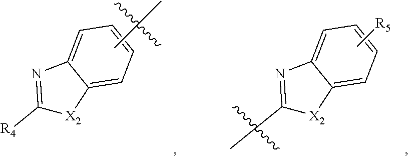

and C₆-C₃₀ aryl or heteroaryl.

In some embodiments, in response to X₃, X₄ and X₅ being carbon respectively, at least one of R₉ or R₁₀ is

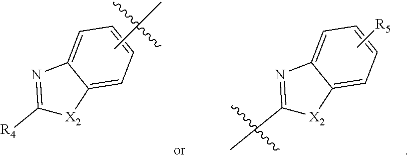

In some embodiments, R₈ is selected from

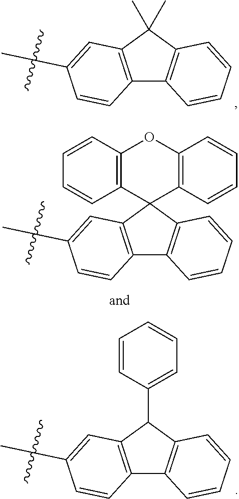

In some embodiments, the structural formula shown in Formula 4 is as follows:

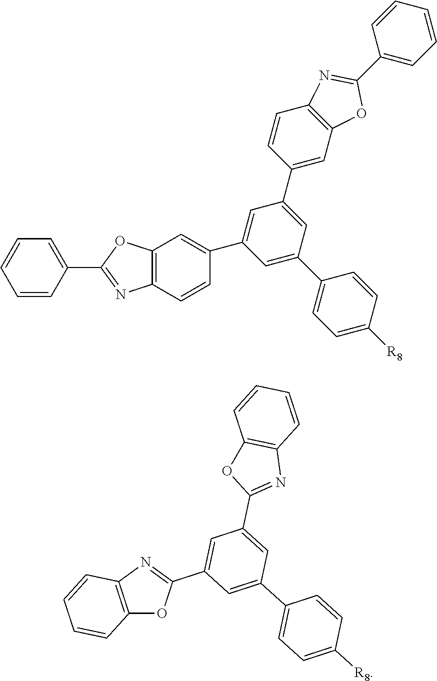

In the light-emitting material and the light-emitting device of the present disclosure, since at least one of substituents R₁, R₂ or R₃ is the M1 group or the M2 group, a LUMO (lowest unoccupied molecular orbital) energy level difference between an N-type material and a doping material can be reduced. Therefore, mobility of electrons in the light-emitting material can be improved, thereby reducing a working voltage or a turn-on voltage. On the other hand, when the light-emitting material is applied to a light-emitting device, a LUMO energy level difference between the N-type material and a hole blocking layer can also be reduced, so that electrons can be easily migrated from the hole blocking layer to the N-type material, thereby reducing the working voltage or the turn-on voltage.

### BRIEF DESCRIPTION OF THE DRAWINGS

FIG. 1 is a schematic diagram of a light-emitting device according to an embodiment of the present disclosure.

FIG. 2 is a light emitting principle diagram of a light-emitting device according to an embodiment of the present disclosure.

Numerals of the drawings are described below: 1 anode: 2 hole injection layer: 3 hole transport layer: 4 electron blocking layer: 5 light-emitting layer: 51 P-type material: 52 N-type material: 53 doping material: 6 hole blocking layer: 7 electron transport layer: 8 electron injection layer: 9 cathode.

### DETAILED DESCRIPTION OF THE EMBODIMENTS

Exemplary embodiments will be described in detail herein, with the illustrations thereof represented in the drawings. When the following descriptions involve the drawings, like numerals in different drawings refer to like or similar elements unless otherwise indicated. The embodiments described in the following examples do not represent all embodiments consistent with the present disclosure. Rather, they are merely examples of apparatuses and methods consistent with some aspects of the present disclosure as detailed in the appended claims.

Terms used herein are used to only describe a particular embodiment rather than limit the present disclosure. Unless otherwise defined, technical terms or scientific terms used in the present disclosure should have general meanings that can be understood by ordinary persons of skill in the art. “First” “second” or the like used in the specification and claims do not represent any sequence, quantity or importance, but distinguish different components. Similarly, “one” or “a” or the like do not represent quantity limitation, but represent at least one. “Multiple” or “a plurality” represents two or more. Unless otherwise stated, the words such as “front”, “rear”, “lower” and/or “upper” are used only for ease of descriptions rather than limited to one position or a spatial orientation. Unless otherwise stated, “include” or “contain” or the like is intended to refer to that an element or object appearing before “include” or “contain” covers an element or object or its equivalents listed after “include” or “contain” and does not preclude other elements or objects. The singular forms such as “a”, “said”, and “the” used in the present disclosure and the appended claims are also intended to include plural forms, unless the context clearly indicates otherwise. It is also to be understood that the term “and/or” as used herein refers to and includes any or all possible combinations of one or more associated listed items.

Embodiments of the present disclosure provide a light-emitting material. The light-emitting material may include a host material and a doping material. The host material may include an N-type material and a P-type material. The N-type material may include a compound with a structural formula shown in Formula 1 or Formula 2:

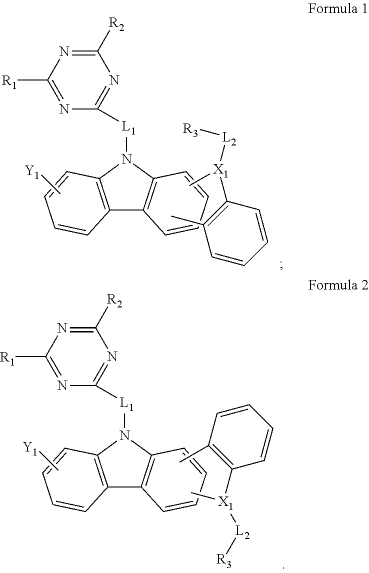

X₁ is selected from nitrogen and carbon: Y₁ is selected from hydrogen, deuterium and tritium: L₁ and L₂ are each independently selected from a single bond and C₆-C₃₀ aryl or heteroaryl. R₁, R₂ and R₃ are each independently selected from an M1 group, an M2 group and C₆-C₃₀ aryl or heteroaryl, and at least one of R₁, R₂ or R; is the M1 group or the M2 group. The M1 group is

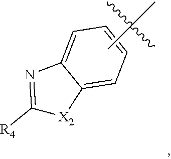

and the M2 group is

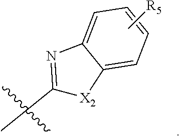

X₂ is selected from sulfur, oxygen and

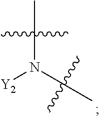

R₄ and R₅ are each independently selected from hydrogen and C₆-C₃₀ aryl or heteroaryl; and Y₂ is selected from C₆-C₃₀ aryl or heteroaryl. The

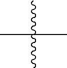

represents a chemical bond.

In the light-emitting material according to embodiments of the present disclosure, since at least one of substituents R₁, R₂ or R; is the M1 group or the M2 group, a LUMO (lowest unoccupied molecular orbital) energy level difference between an N-type material and a doping material can be reduced. Therefore, mobility of electrons in the light-emitting material can be improved, thereby reducing a working voltage or a turn-on voltage. On the other hand, when the light-emitting material is applied to a light-emitting device, a LUMO energy level difference between the N-type material and a hole blocking layer can also be reduced, so that electrons can be easily migrated from the hole blocking layer to the N-type material, thereby reducing the working voltage or the turn-on voltage.

X₁ is selected from nitrogen and carbon, that is, X₁ may be nitrogen or carbon. For example, X₁ is nitrogen. Y₁ is selected from hydrogen, deuterium and tritium. Hydrogen may also be referred to as “protium”. L₁ is selected from a single bond and C₆-C₃₀ aryl or heteroaryl, where the C₆-C₃₀ aryl may be phenyl, biphenyl, naphthyl or the like. In other embodiments, L₁ may be selected from C₁-C₁₀ alkyl, where the C₁-C₁₀ alkyl may be a linear alkyl, which is not limited thereto, and may further be cycloalkyl or the like. L₂ is selected from a single bond and C₆-C₃₀ aryl or heteroaryl, where the C₆-C₃₀ aryl may be phenyl, biphenyl, naphthyl or the like. In other embodiments, L₂ may be selected from C₁-C₁₀ alkyl, where the C₁-C₁₀ alkyl may be a linear alkyl, which is not limited thereto, and may further be cycloalkyl. L₁ and L₂ may be the same or different. For example, L and L₂ are the same, and both are single bonds.

R₁, R₂ and R₃ are each independently selected from an M1 group, an M2 group and C₆-C₃₀ aryl or heteroaryl, where the C₆-C₃₀ aryl may be phenyl, biphenyl, naphthyl or the like. R₁, R₂ and R; may be the same as or different from each other. In some embodiments, at least one of R₁, R₂ or R₃ is the M1 group or the M2 group. For example, in R₁, R₂ and R₃, only R₁ is the M1 group or the M2 group. For another example, in R₁, R₂ and R₃, only R₃ is the M1 group or the M2 group.

The M1 group may be

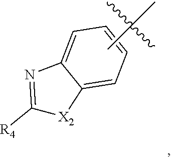

and the M2 group may be

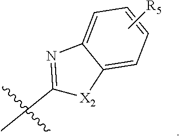

X₂ is selected from sulfur, oxygen and

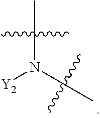

Y₂ is selected from C₆-C₃₀ aryl or heteroaryl, where the C₆-C₃₀ aryl or heteroaryl may be phenyl, biphenyl, triphenyl, fluorenyl, dimethylfluorenyl, diphenylfluorenyl, spirofluorenyl, diphenylfuryl, dibenzothiophenyl or the like. R₄ and R₅ are each independently selected from hydrogen and C₆-C₃₀ aryl or heteroaryl, where the C₆-C₃₀ aryl may be phenyl, biphenyl, naphthyl or the like. In other embodiments, the M1 group may be

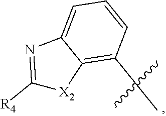

and the M2 group may be

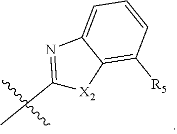

The structural formula shown in Formula 1 may be

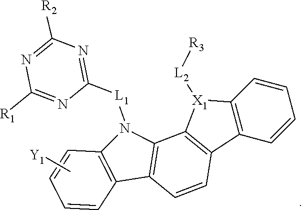

The structural formula shown in Formula 2 may be

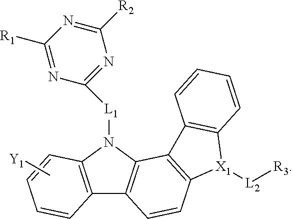

In some embodiments, the structural formula shown in Formula 1 may be as follows:

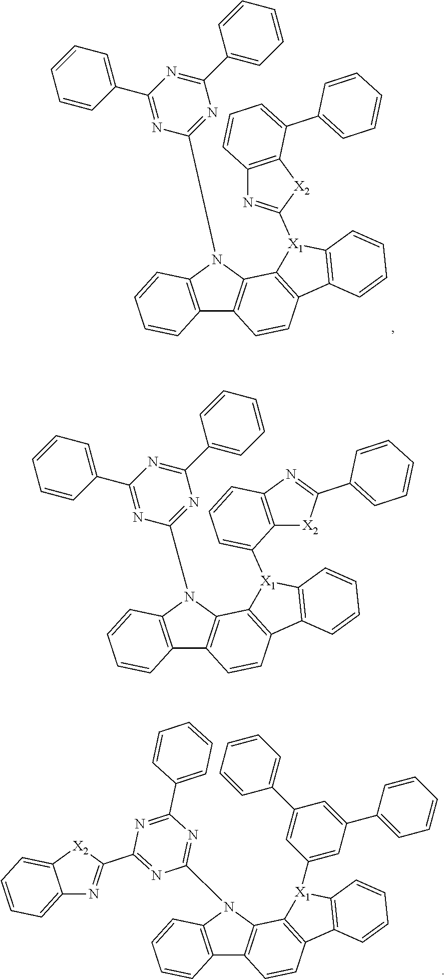

In some embodiments, the compound with the structural formula shown in Formula 1 may be as follows:

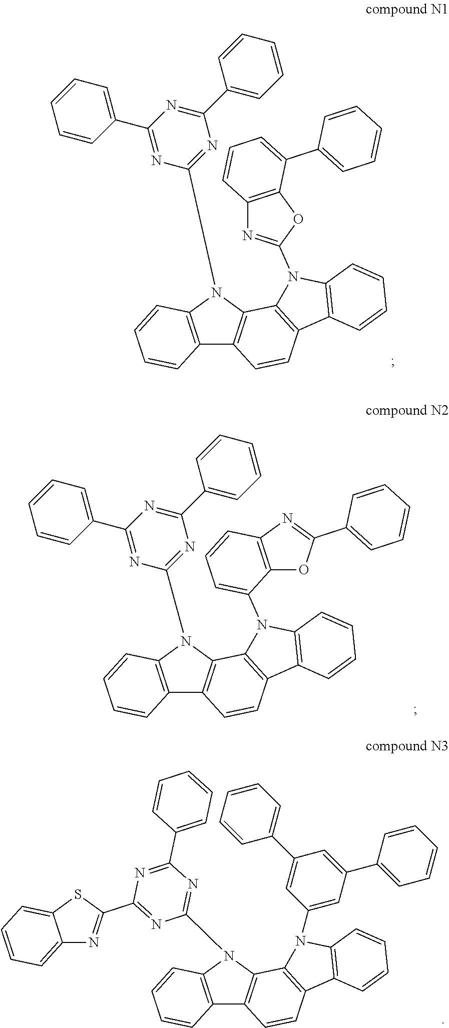 [SMILES: c1ccc(-c2nc(-c3ccccc3)nc(-n3c4ccccc4c4ccc5c6ccccc6n(-c6nc7cccc(-c8ccccc8)c7o6)c5c43)n2)cc1 ; c1ccc(-c2nc(-c3ccccc3)nc(-n3c4ccccc4c4ccc5c6ccccc6n(-c6cccc7nc(-c8ccccc8)oc67)c5c43)n2)cc1 ; c1ccc(-c2cc(-c3ccccc3)cc(-n3c4ccccc4c4ccc5c6ccccc6n(-c6nc(-c7ccccc7)nc(-c7nc8ccccc8s7)n6)c5c43)c2)cc1]

Method of synthesizing compound N1:

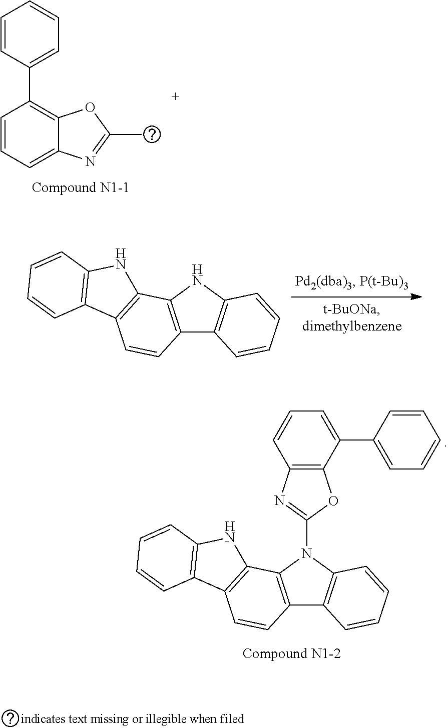 [SMILES: Cc1nc2cccc(-c3ccccc3)c2o1 ; c1ccc2c(c1)[nH]c1c2ccc2c3ccccc3[nH]c21 ; c1ccc(-c2cccc3nc(-n4c5ccccc5c5ccc6c7ccccc7[nH]c6c54)oc23)cc1]

To a three-necked flask equipped with a thermometer, a mechanical stirrer and a spherical condenser tube was introduced N₂ (0.1 L/min) for 18 min and added compound N1-1 (26.3 g, 114.5 mmol), indolo[2,3-A]carbazole (35.3 g, 137.6 mmol), Pd₂(dba)₃ (2.1 g, 2.3 mmol), tri-tert-butylphosphine (0.92 g, 4.6 mmol), sodium tert-butoxide (27.5 g, 286.2 mmol) and dimethylbenzene (500 mL). The solution was heated to 138° C.-142° C. under stirring, and then refluxed for 12 h. After the reaction was finished, the reaction solution was cooled to room temperature. The reaction solution was washed with water, and the organic phase was separated. The organic phase was dried with anhydrous magnesium sulfate, and filtered to remove the solvent. The crude product was recrystallized with a dichloromethane/ethanol system to obtain an intermediate compound N1-2 (40.0 g, 78%) as a white solid.

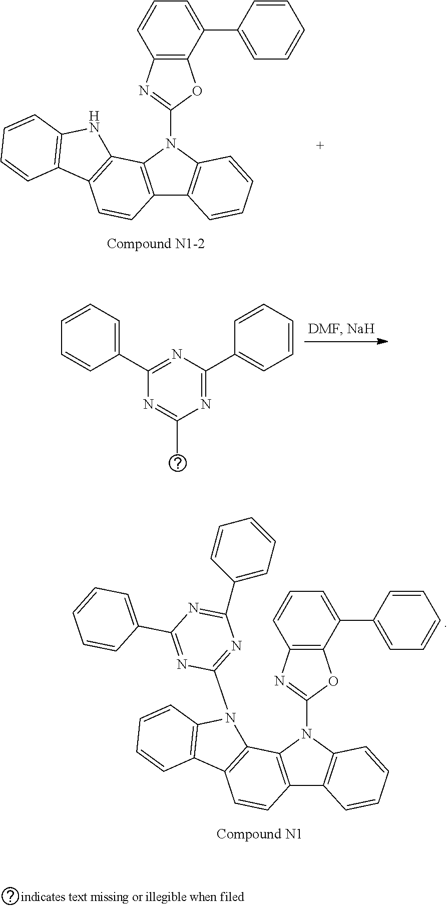 [SMILES: c1ccc(-c2cccc3nc(-n4c5ccccc5c5ccc6c7ccccc7[nH]c6c54)oc23)cc1 ; Cc1nc(-c2ccccc2)nc(-c2ccccc2)n1 ; c1ccc(-c2nc(-c3ccccc3)nc(-n3c4ccccc4c4ccc5c6ccccc6n(-c6nc7cccc(-c8ccccc8)c7o6)c5c43)n2)cc1]

To a three-necked flask equipped with a mechanical stirrer, a thermometer and a spherical condenser tube was introduced N2 (0.1 L/min) for 18 min and added the intermediate compound N1-2 (17.0 g, 38.0 mmol), 2-chloro-4,6-diphenyl-1,3,5-triazine (35.3 g, 137.6 mmol) and DMF (200 mL). The solution was cooled to 0° C. After NaH (1.0 g, 41.8 mmol) was added, the system changed from white to yellow. When the solution was heated to room temperature, a solid was precipitated, and the reaction was finished. The reaction solution was washed with water, and filtered to obtain a solid product, which was eluted with a small amount of ethanol. And the crude product was recrystallized with methylbenzene to obtain the compound N1 (16.6 g, 64%).

The P-type material in the host material includes a compound with a structural formula shown in Formula 3:

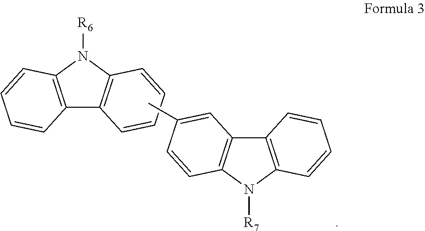

R₆ and R₇ are each independently C₆-C₃₀ aryl or heteroaryl, where the C₆-C₃₀ aryl or heteroaryl may be phenyl, biphenyl, triphenyl, fluorenyl, dimethylfluorenyl, diphenylfluorenyl, spirofluorenyl, diphenylfuryl, dibenzothiophenyl or the like. For example, R₆ and R₇ are each independently selected from structural formulas as follows:

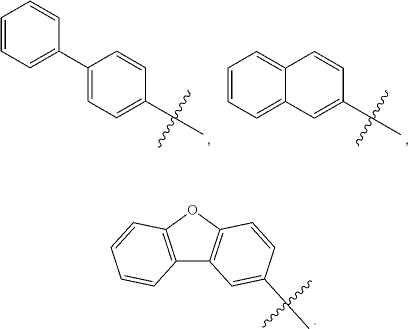 [SMILES: *c1ccc(-c2ccccc2)cc1 ; *c1ccc2ccccc2c1 ; *c1ccc2oc3ccccc3c2c1]

In some embodiments, the compound with the structural formula shown in Formula 3 may be as follows:

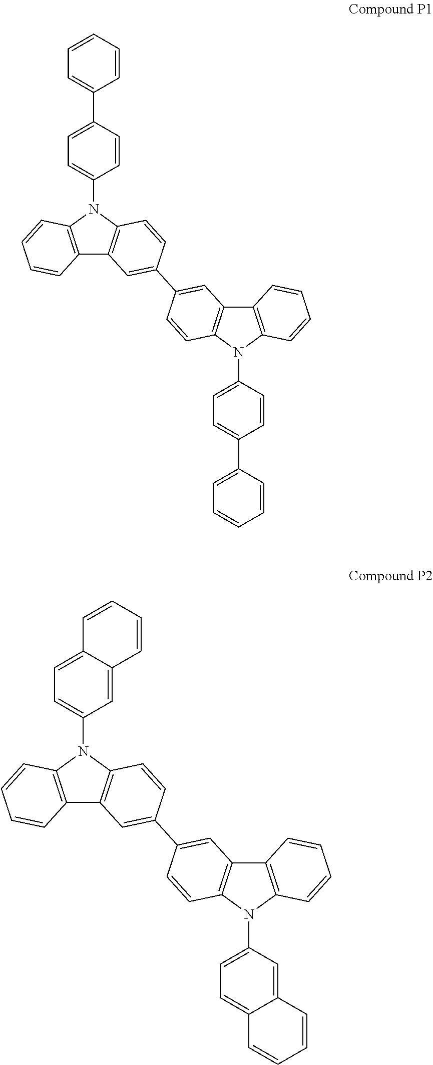 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5ccc6ccccc6c5)ccc43)ccc2c1]

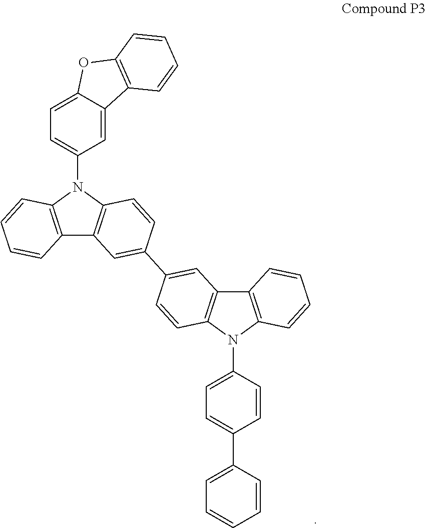 [SMILES: c1ccc(-c2ccc(-n3c4ccccc4c4cc(-c5ccc6c(c5)c5ccccc5n6-c5ccc6oc7ccccc7c6c5)ccc43)cc2)cc1]

In the light-emitting material, a ratio between the amount of substance of the P-type material and the amount of substance of the N-type material may be 5:5 to 7:3, for example, 5:5, 6:4, 7:3 or the like, which is not limited thereto in the present disclosure.

The doping material may be a phosphorescent material. In some embodiments, the doping material may be a green phosphorescent material. The phosphorescent material may be a phosphor electroluminescent material. A mass ratio of the doping material to the host material may be 10/100 to 15/100, for example, 10/100, 11/100, 12/100, 13/100, 14/100, 15/100 or the like.

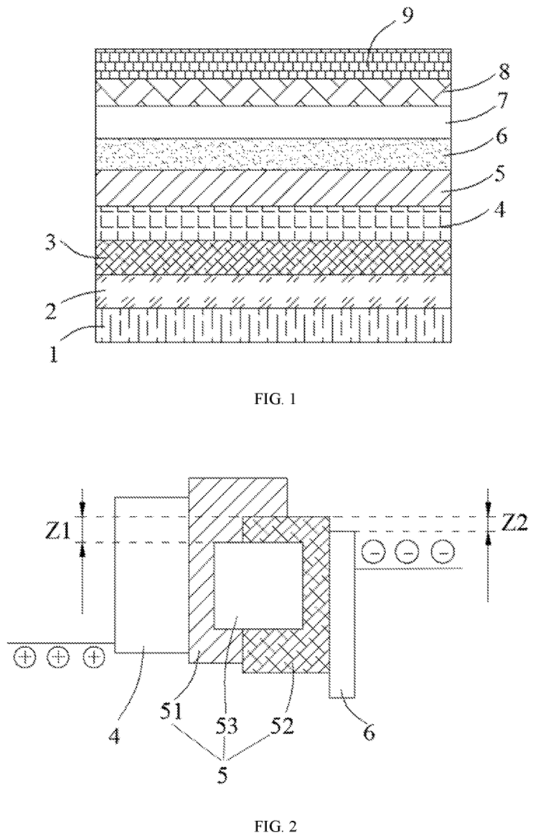

The embodiments of the present disclosure further provide a light-emitting device. As shown in FIG. 1, the light-emitting device may include an anode 1, a cathode 9 and a light-emitting layer 5.

The anode 1 and the cathode 9 are arranged oppositely to each other. The light-emitting layer 5 is disposed between the anode 1 and the cathode 9. The light-emitting layer 5 includes the light-emitting material described in any one of the above embodiments.

The light-emitting material included in the light-emitting device of the embodiments of the present disclosure is the same as the light-emitting material of the above-mentioned embodiments of light-emitting material, and therefore has the same beneficial effects, which is not repeated herein in the present disclosure.

The anode 1 may include a material with a large work function. Examples of the material of the anode 1 may include: a metal such as nickel, platinum, vanadium, chromium, copper, zinc and gold or alloys thereof; a metal oxide such as zinc oxide, indium oxide, indium tin oxide (ITO) and indium zinc oxide (IZO); a combination of metal and oxide such as ZnO:Al or SnO₂:Sb; or a conductive polymer such as poly(3-methylthiophene), poly[3,4-(ethylene-1,2-dioxy)]thiophene (PEDT), polypyrrole and polyaniline, which is not limited thereto. Indium tin oxide (ITO) is preferably as the anode 1.

The cathode 9 may include a material with a small work function. Examples of the material of the cathode 9 may include: a metal such as magnesium, calcium, sodium, potassium, titanium, indium, yttrium, lithium, gadolinium, aluminum, silver, tin and lead or alloys thereof; or a multilayer material such as LiF/Al, Liq/Al, LiO₂/Al, LiF/Ca, LiF/Al and BaF₂/Ca, which is not limited thereto. A metal electrode including an Mg—Ag alloy is preferably as the cathode 9.

The light-emitting device may further include a hole injection layer 2 and a hole transport layer 3. The hole injection layer 2 may be disposed between the anode 1 and the light-emitting layer 5, and the hole transport layer 3 may be disposed between the hole injection layer 2 and the light-emitting layer 5. In some embodiments, the light-emitting device may further include an electron blocking layer 4. The electron blocking layer 4 may be disposed between the hole transport layer 3 and the light-emitting layer 5.

In some embodiments, the light-emitting device may further include an electron injection layer 8, an electron transport layer 7, and a hole blocking layer 6. The electron injection layer 8 may be disposed between the cathode 9 and the light-emitting layer 5, the electron transport layer 7 may be disposed between the electron injection layer 8 and the light-emitting layer 5, and the hole blocking layer 6 may be disposed between the electron transport layer 7 and the light-emitting layer 5. The hole blocking layer 6 may include a compound with a structural formula shown in Formula 4:

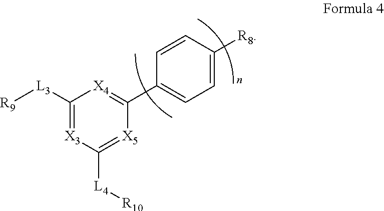

n is a positive integer greater than or equal to 1 and less than or equal to 5, for example, 1, 2, 3, 4, 5, etc. R₉ and R₁₀ are each independently selected from the M1 group, the M2 group and C₆-C₃₀ aryl or heteroaryl, where the C₆-C₃₀ aryl or heteroaryl may be phenyl, biphenyl, triphenyl, fluorenyl, dimethylfluorenyl, diphenylfluorenyl, spirofluorenyl, diphenylfuryl, dibenzothiophenyl or the like. R₉ and R₁₀ may be the same or different.

X₃, X₄ and X₅ are each independently selected from carbon and nitrogen. X₃, X₄ and X₅ may be the same as each other, which is not particularly limited in the present disclosure. L₃ and L₄ are each independently selected from a single bond, C₆-C₃₀ aryl or heteroaryl, and C₁-C₁₀ alkyl, where the C₆-C₃₀ aryl or heteroaryl may be phenyl, biphenyl, triphenyl, fluorenyl, dimethylfluorenyl, diphenylfluorenyl, spirofluorenyl, diphenylfuryl, dibenzothiophenyl or the like. L; and L₄ may be the same or different.

In some embodiments, X₃, X₄ and X₅ are carbon respectively, and at least one of R₉ and R₁₀ is the M1 group or the M2 group. In other embodiments, at least one of X₃, X₄ or X₅ is nitrogen, and R₉ and R₁₀ are each independently selected from phenyl, biphenyl, naphthyl and phenanthrenyl.

R₈ is selected from hydrogen and C₆-C₃₀ aryl or heteroaryl, where the C₆-C₃₀ aryl or heteroaryl may be phenyl, biphenyl, triphenyl, fluorenyl, dimethylfluorenyl, diphenylfluorenyl, spirofluorenyl, diphenylfuryl, dibenzothiophenyl or the like.

In some embodiments, R₈ is selected from

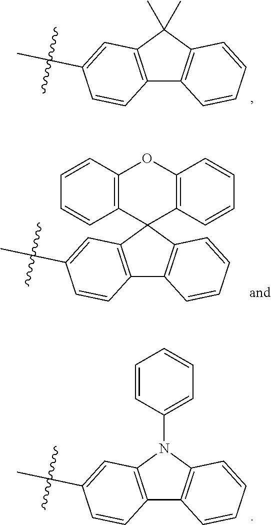 [SMILES: *c1ccc2c(c1)C(C)(C)c1ccccc1-2 ; *c1ccc2c(c1)C1(c3ccccc3Oc3ccccc31)c1ccccc1-2 ; *c1ccc2c3ccccc3n(-c3ccccc3)c2c1]

In some embodiments, the structural formula shown in Formula 4 may be as follows:

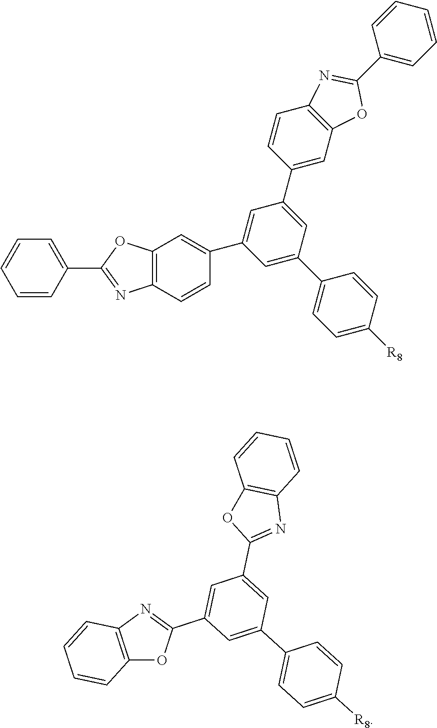

For example, the compound with the structural formula shown in Formula 4 may be as follows:

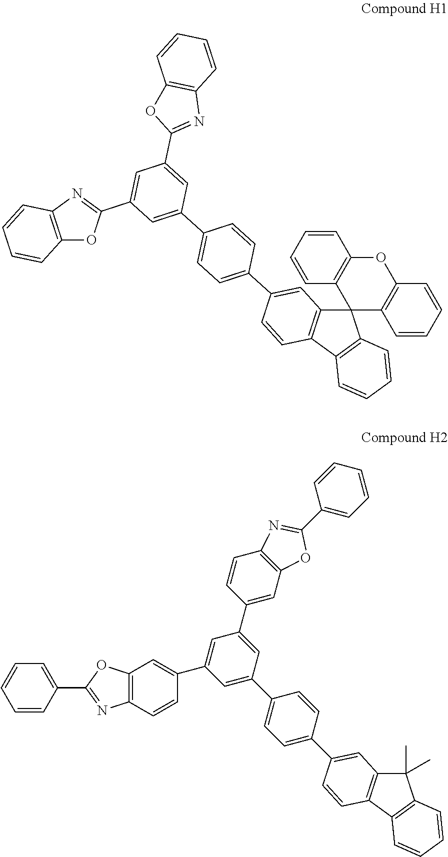 [SMILES: c1ccc2c(c1)Oc1ccccc1C21c2ccccc2-c2ccc(-c3ccc(-c4cc(-c5nc6ccccc6o5)cc(-c5nc6ccccc6o5)c4)cc3)cc21 ; CC1(C)c2ccccc2-c2ccc(-c3ccc(-c4cc(-c5ccc6nc(-c7ccccc7)oc6c5)cc(-c5ccc6nc(-c7ccccc7)oc6c5)c4)cc3)cc21]

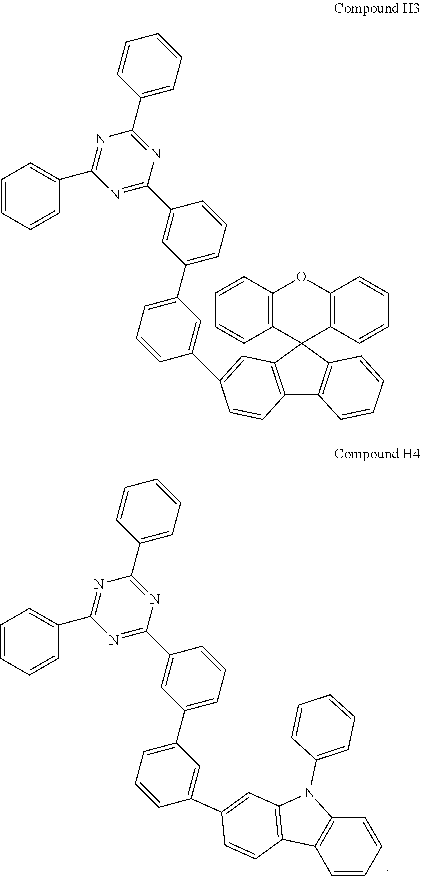 [SMILES: c1ccc(-c2nc(-c3ccccc3)nc(-c3cccc(-c4cccc(-c5ccc6c(c5)C5(c7ccccc7Oc7ccccc75)c5ccccc5-6)c4)c3)n2)cc1 ; c1ccc(-c2nc(-c3ccccc3)nc(-c3cccc(-c4cccc(-c5ccc6c7ccccc7n(-c7ccccc7)c6c5)c4)c3)n2)cc1]

Method of synthesizing compound H1:

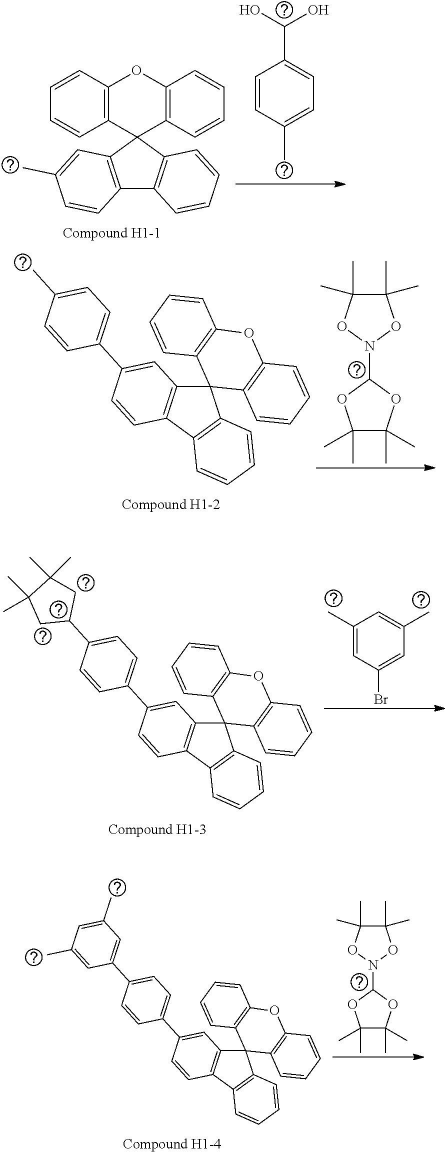

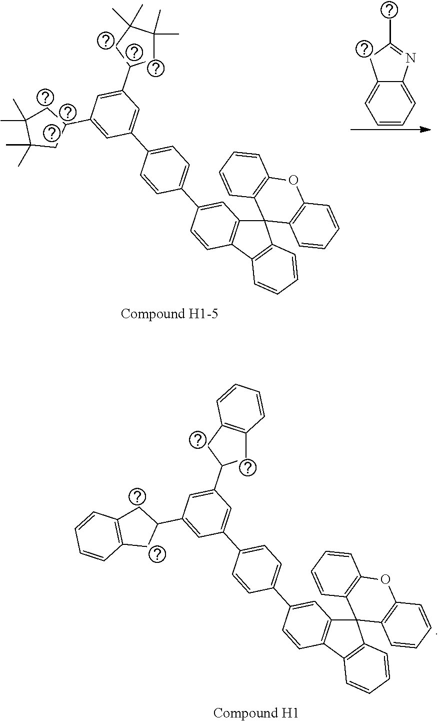

(1) Under N2 atmosphere, tricyclohexylphosphane (0.560 g, 2.0 mmol) and tris(dibenzylideneacetone) dipalladium (0.906 g, 1.0 mmol) were added to a mixed solution of 2-bromospiro[fluorene-9,9′-xanthene] (compound H1-1, 24.6 g. 60 mmol), 4-chlorophenylboronic acid (7.82 g, 50 mmol), dioxane (160 ml) and sodium carbonate solution (2.50) M, 80 ml), and refluxed overnight. The reaction solution was cooled to room temperature and extracted with ethyl acetate. The organic layer was dried with anhydrous magnesium sulfate, filtered, distilled to remove the solvent, and then separated by silica gel column. The eluent was dichloromethane and n-hexane (2:1), and 18.17 g of compound H1-2 was obtained with a yield of 82%.

(2) Under N2 atmosphere, bis(triphenylphosphine) palladium dichloride (0.490 g, 0.70 mmol) was added to a mixed solution of bis(pinacolato) diboron (6.66 g, 42.0 mmol), compound H1-2 (15.58 g, 35.0 mmol), potassium acetate (10.30 g, 105.0 mmol) and tetrahydrofuran (460) ml), and refluxed for 4 h. The reaction solution was cooled to room temperature, distilled to remove tetrahydrofuran, and added chloroform and distilled water. The organic layer was separated, and the water layer was extracted with chloroform. The organic layer was dried with anhydrous magnesium sulfate, filtered, distilled under reduced pressure to remove the solvent, and then separated by silica gel column. The eluent was dichloromethane and petroleum ether (3:1), and 16.08 g of compound H1-3 was obtained with a yield of 86%.

(3) Under N2 atmosphere, trtrakis (triphenylphosphine) palladium (0.324 g, 0.28 mmol) was added to a mixed solution of 1-bromo-3,5-dichlorobenzene (6.32 g, 28 mmol), compound H1-3 (14.96 g, 28 mmol), sodium carbonate solution (4 M, 49 ml), ethanol (50 ml) and methylbenzene (500 ml), and refluxed under stirring overnight. The reaction solution was cooled to room temperature, and extracted with chloroform. The organic layer was dried with anhydrous magnesium sulfate, filtered, distilled under reduced pressure to remove the solvent, and then separated by silica gel column. The eluent was ethyl acetate and n-hexane (2:1), and 14.03 g of compound H1-4 was obtained with a yield of 84%.

(4) Under N2 atmosphere, tris(dibenzylideneacetone) dipalladium (0.732 g. 0.8 mmol) and tricyclohexylphosphane (0.448 g, 1.6 mmol) were added to a mixed solution of compound H1-4 (11.08 g. 20 mmol), bis(pinacolato) diboron (10.16 g, 40 mmol), sodium carbonate solution (2.50 M, 64 ml) and dioxane (130 ml), and refluxed overnight. The reaction solution was cooled to room temperature, and extracted with dichloromethane. The organic layer was dried with anhydrous magnesium sulfate, filtered, distilled under reduced pressure to remove the solvent, and then separated by silica gel column. The eluent was dichloromethane and n-hexane (5:2), and 11.50 g of compound H1-5 was obtained with a yield of 78%.

(5) Under N2 atmosphere, 1, l′-bis(diphenylphosphino) ferrocene palladium (II) dichloride (1.464 g, 0.2 mmol) was added to a mixed solution of compound H1-5 (7.36 g, 10 mmol), 2-chlorobenzoxazole (3.68 g, 24 mmol), potassium phosphate solution (4 M, 20 ml) and methylbenzene (100 ml), and refluxed for one day. The reaction solution was cooled to room temperature, and extracted with dichloromethane. The organic layer was dried with anhydrous magnesium sulfate, filtered, distilled under reduced pressure to remove the solvent, and then separated by silica gel column. The eluent was dichloromethane and ethanol (3:1), and 6.04 g of compound H1 was obtained with a yield of 84%.

As shown in FIG. 2, in the light-emitting device of the present disclosure, since at least one of substituents R₁, R₂ or R₃ is the M1 group or the M2 group, a LUMO (lowest unoccupied molecular orbital) energy level difference Z1 between an N-type material 52 and a doping material 53 can be reduced. Therefore, mobility of electrons in the light-emitting material can be improved, thereby reducing a working voltage or a turn-on voltage. On the other hand, when the light-emitting material is applied to a light-emitting device, a LUMO energy level difference Z2 between the N-type material 52 and a hole blocking layer 6 can also be reduced, so that electrons can be easily migrated from the hole blocking layer 6 to the N-type material 52, thereby reducing the working voltage or the turn-on voltage.

Examples of manufacturing the light-emitting device of this embodiment are provided below, so as to specifically describe the structure of the light-emitting device.

### Example 1

A glass plate with ITO was ultrasonic treated in a cleaning agent, rinsed in deionized water, ultrasonic deoiled in an acetone-ethanol mixed solvent, and baked in a clean environment until water was completely removed. The glass plate with ITO is used as an anode of a light-emitting device. On a side of the anode, a hole injection layer, a hole transport layer, an electron blocking layer, a light-emitting layer, a hole blocking layer, an electron transport layer, an electron injection layer and a cathode were sequentially evaporated. A thickness of the electron blocking layer may be 30 nm, a thickness of the light-emitting layer may be 30 nm, and a thickness of the hole blocking layer may be 5 nm. In the light-emitting layer, a mass ratio of the host material to the doping material may be 100:12. In the host material, a ratio between the amount of substance of the P-type material and the amount of substance of the N-type material is 6:4.

The N-type material in the host material was formed by the above compound N1, the P-type material in the host material was formed by the above compound P1, and the hole blocking layer was formed by the above compound H1.

### Example 2

A light-emitting device was manufactured by the same manufacturing method as that of Example 1. And the difference lies in that the N-type material in the host material was formed by the above compound N2, the P-type material in the host material was formed by the above compound P2, and the hole blocking layer was formed by the above compound H2.

### Example 3

A light-emitting device was manufactured by the same manufacturing method as that of Example 1. And the difference lies in that the N-type material in the host material was formed by the above compound N3, the P-type material in the host material was formed by the above compound P3, and the hole blocking layer was formed by the above compound H3.

### Example 4

A light-emitting device was manufactured by the same manufacturing method as that of Example 1. And the difference lies in that a ratio between the amount of substance of the P-type material and the amount of substance of the N-type material is 5:5.

### Example 5

A light-emitting device was manufactured by the same manufacturing method as that of Example 1. And the difference lies in that a ratio between the amount of substance of the P-type material and the amount of substance of the N-type material is 7:3.

### Comparative Example 1

A light-emitting device was manufactured by the same manufacturing method as that of Example 1. And the difference lies in that the N-type material in the host material was formed by compound N4, the P-type material in the host material was formed by compound P4, and the hole blocking layer was formed by the above compound H4. The structural formulas of the compound N4 and the compound P4 are as follows:

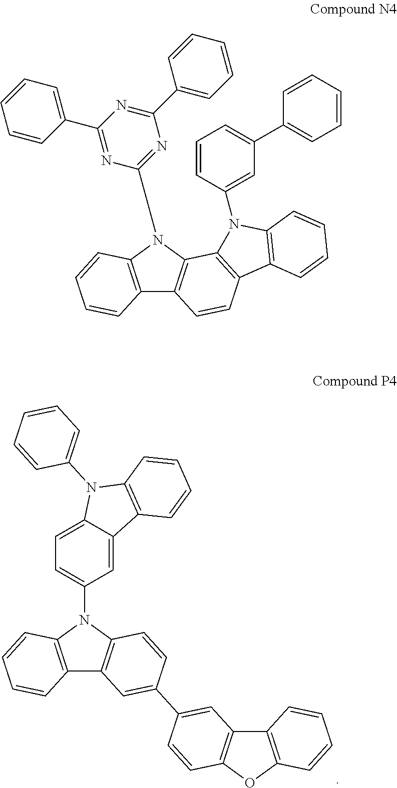 [SMILES: c1ccc(-c2cccc(-n3c4ccccc4c4ccc5c6ccccc6n(-c6nc(-c7ccccc7)nc(-c7ccccc7)n6)c5c43)c2)cc1 ; c1ccc(-n2c3ccccc3c3cc(-n4c5ccccc5c5cc(-c6ccc7oc8ccccc8c7c6)ccc54)ccc32)cc1]

The physical property data of the compounds used to manufacture the above-mentioned light-emitting devices are shown in Table 1.

TABLE 1

| | | | | | μh (hole | μe (electron |
|---|---|---|---|---|---|---|
| | HOMO/ | LUMO/ | T1/ | Tg/ | mobility) | mobility) |
| | eV | eV | eV | ° C. | cm²/v · s | cm²/v · s |
| P1 | 5.43 | 2.11 | 2.69 | 108 | 8.2 × 10⁻⁶ | — |
| P2 | 5.47 | 2.14 | 2.67 | 116 | 5.4 × 10⁻⁵ | — |
| P3 | 5.41 | 2.10 | 2.65 | 115 | 4.1 × 10⁻⁵ | — |
| P4 | 5.44 | 2.08 | 2.66 | 96 | 3.6 × 10⁻⁶ | — |
| N1 | 5.51 | 2.40 | 2.62 | 120 | — | 5.6 × 10⁻⁶ |
| N2 | 5.54 | 2.38 | 2.61 | 116 | — | 2.3 × 10⁻⁵ |
| N3 | 5.56 | 2.42 | 2.58 | 118 | — | 8.4 × 10⁻⁶ |
| N4 | 5.54 | 2.58 | 2.54 | 109 | — | 1.4 × 10⁻⁶ |
| HB1 | 6.20 | 2.68 | 2.65 | 126 | — | 7.2 × 10⁻⁸ |
| HB2 | 6.17 | 2.71 | 2.63 | 128 | — | 6.6 × 10⁻⁸ |
| HB3 | 6.14 | 2.74 | 2.64 | 122 | — | 2.2 × 10⁻⁸ |
| HB4 | 6.28 | 2.84 | 2.55 | 112 | — | 8.6 × 10⁻⁹ |

In the present disclosure, the performance of the manufactured light-emitting devices was tested, and the results are shown in Table 2.

TABLE 2

| | working | turn-on | | | | |
|---|---|---|---|---|---|---|
| | oltage | voltage | | | | |
| | V | Von | Cd/A | CIE x | CIE y | LT95 (h) |
| Example 1 | 90% | 89% | 118% | 0.25 | 0.72 | 105% |
| Example 2 | 88% | 87% | 112% | 0.25 | 0.72 | 108% |
| Example 3 | 91% | 89% | 113% | 0.25 | 0.72 | 100% |
| Example 4 | 85% | 84% | 112% | 0.25 | 0.72 | 98% |
| Example 5 | 98% | 96% | 124% | 0.25 | 0.72 | 112% |
| Comparative | 100% | 100% | 100% | 0.25 | 0.72 | 100% |
| example 1 | | | | | | |

In Table 2, with reference to the data of Comparative Example 1, the data of voltage, efficiency and life are set to 100%. It can be seen that in Example 4, Example 1 and Example 5, the compound P1 and the compound N1 are used, and rations between the amount of substance of the compound P1 and the amount of substance of the compound N1 are 5:5, 6:4, and 7:3 in order: with the increase of the ratio of the amount of substance, the efficiency is gradually improved, and the device voltage is also gradually increased: but respective device voltages are lower than that in Comparative Example 1. Therefore, in Examples 1-5 of the present disclosure, not only the efficiency is improved, but also the device voltage is reduced.

The above descriptions are made merely to preferred embodiments of the present disclosure rather than intended to limit the present disclosure in any manner. Although the present disclosure is made with preferred embodiments as above, these preferred embodiments are not used to limit the present disclosure. Those skilled in the art may make some changes or modifications to the technical contents of the present disclosure as equivalent embodiments without departing from the scope of the technical solution of the present disclosure. Any simple changes, equivalent changes or modifications made to the above embodiments based on the technical essence of the present disclosure without departing from the contents of the technical solution of the present disclosure shall all fall within the scope of protection of the present disclosure.

1. A light-emitting material, comprising:
a host material, and
a doping material,
wherein the host material comprises an N-type material and a P-type material, and the N-type material comprises a compound with a structural formula shown in Formula 1 or Formula 2: 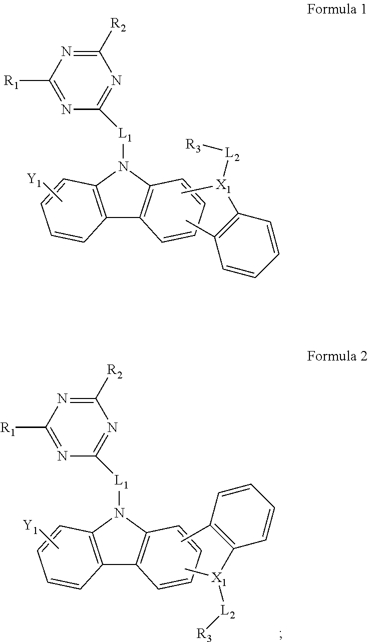
wherein X₁ is selected from nitrogen and carbon;
Y₁ is selected from hydrogen, deuterium and tritium;
L₁ and L₂ are each independently selected from a single bond and C₆-C₃₀ aryl or heteroaryl;
R₁, R₂ and R₃ are each independently selected from 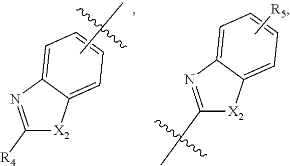
and C₆-C₃₀ aryl or heteroaryl;
at least one of R₁, R₂ or R₃ is the 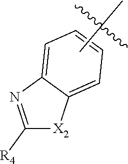
or the 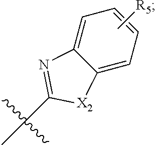
X₂ is selected from sulfur, oxygen and 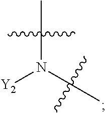
R₄ and R₅ are each independently selected from hydrogen and C₆-C₃₀ aryl or heteroaryl;
Y₂ is selected from C₆-C₃₀ aryl or heteroaryl.

2. The light-emitting material according to claim 1, wherein,
the

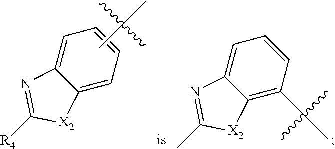

3. The light-emitting material according to claim 1, wherein R⁴ and R₅ are each independently selected from hydrogen and phenyl.

4. The light-emitting material according to claim 1, wherein,
the structural formula shown in Formula 1 is as follows:

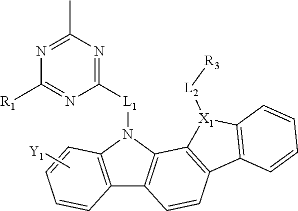

5. The light-emitting material according to claim 1, wherein the structural formula shown in Formula 1 is as follows:

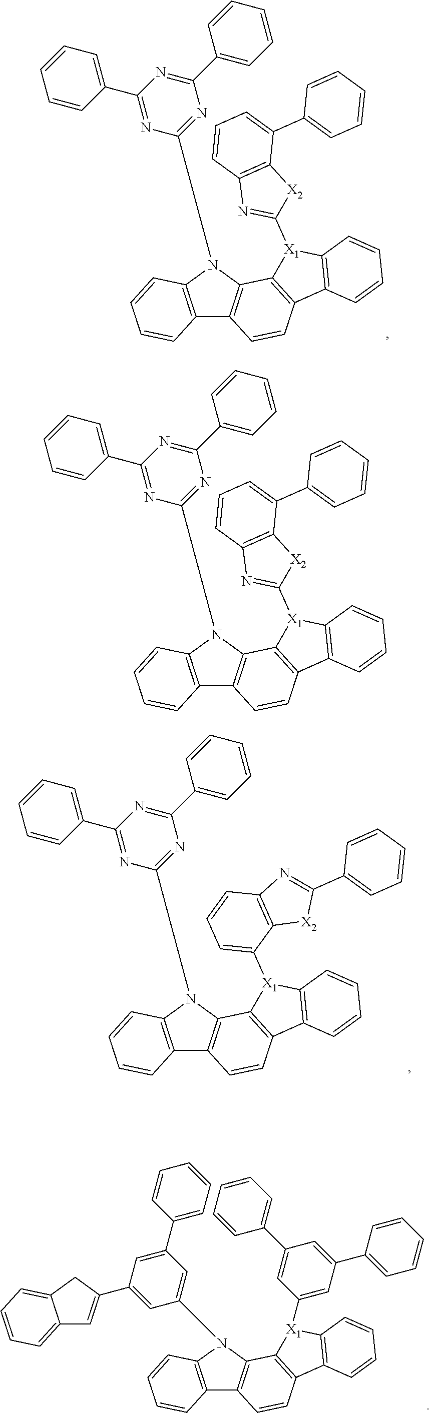

6. The light-emitting material according to claim 1, wherein the doping material is a phosphorescent material.

7. The light-emitting material according to claim 6, wherein the doping material is a green phosphorescent material.

8. The light-emitting material according to claim 1, wherein the P-type material comprises a compound with a structural formula shown in Formula 3: 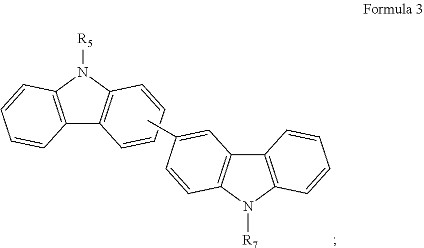
wherein R₆ and R₇ are each independently C₆-C₃₀ aryl or heteroaryl.

9. The light-emitting material according to claim 8, wherein R₆ and R₇ are each independently selected from structural formulas as follows:

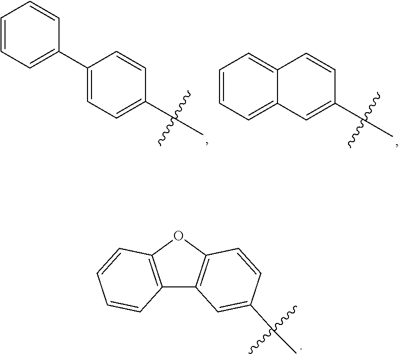 [SMILES: *c1ccc(-c2ccccc2)cc1 ; *c1ccc2ccccc2c1 ; *c1ccc2oc3ccccc3c2c1]

10. The light-emitting material according to claim 8, wherein the structural formula shown in Formula 3 is as follows:

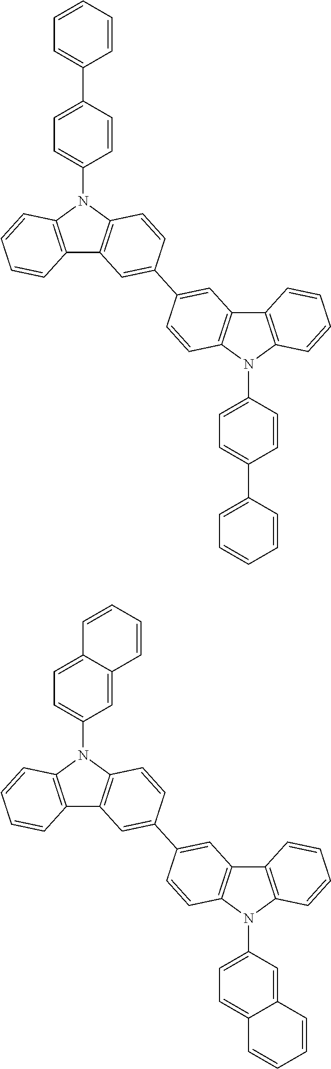 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5ccc6ccccc6c5)ccc43)ccc2c1]

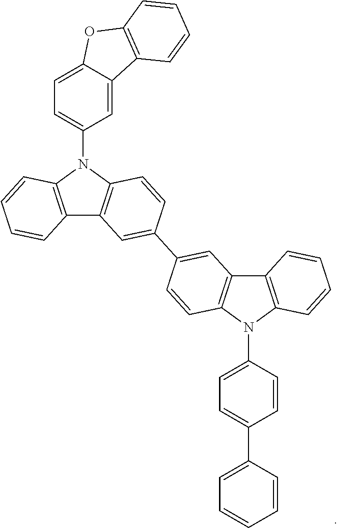 [SMILES: c1ccc(-c2ccc(-n3c4ccccc4c4cc(-c5ccc6c(c5)c5ccccc5n6-c5ccc6oc7ccccc7c6c5)ccc43)cc2)cc1]

11. A light-emitting device, comprising:
an anode and a cathode arranged opposite to each other; and
a light-emitting layer, disposed between the anode and the cathode, wherein the light-emitting layer comprises the light-emitting material according to any one of claim 1.

12. The light-emitting device according to claim 11, further comprising:
an electron transport layer, disposed between the light-emitting layer and the cathode; and
a hole blocking layer, disposed between the electron transport layer and the light-emitting layer, wherein the hole blocking layer comprises a compound with a structural formula shown in Formula 4: 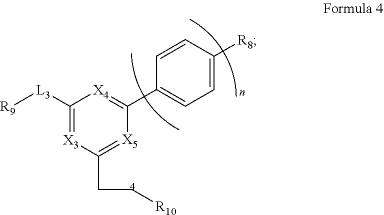
wherein n is a positive integer greater than or equal to 1 and less than or equal to 5;
X₃, X₄ and X₅ are each independently selected from carbon and nitrogen;
L₃ and La are each independently selected from a single bond and C₆-C₃₀ aryl or heteroaryl;
R₈ is selected from hydrogen, C₆-C₃₀ aryl or heteroaryl;
R₉ and R₁₀ are each independently selected from 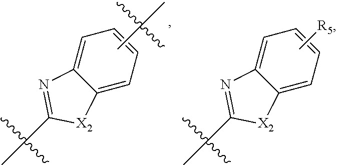
and C₆-C₃₀ aryl or heteroaryl.

13. The light-emitting device according to claim 12, wherein in response to X₃, X₄ and X₅ being carbon respectively, at least one of R₉ and R₁₀ is

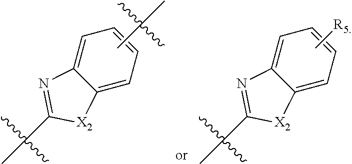

14. The light-emitting device according to claim 12, wherein R₈ is selected from:

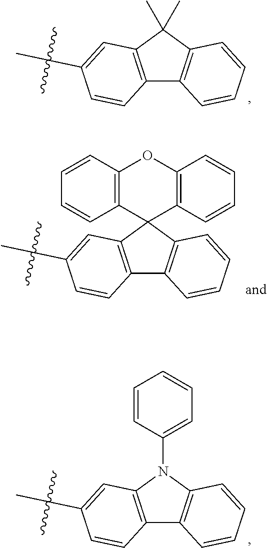 [SMILES: *c1ccc2c(c1)C(C)(C)c1ccccc1-2 ; *c1ccc2c(c1)C1(c3ccccc3Oc3ccccc31)c1ccccc1-2 ; *c1ccc2c3ccccc3n(-c3ccccc3)c2c1]

15. The light-emitting device according to claim 12, wherein the structural formula shown in Formula 4 is as follows:

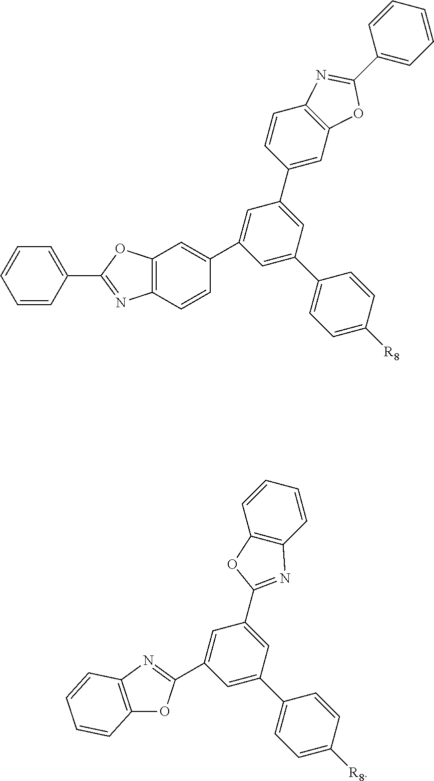

16. The light-emitting material according to claim 1, wherein
the

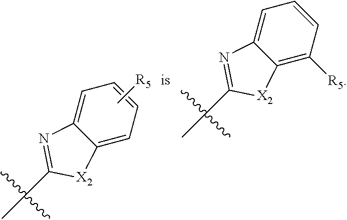

17. The light-emitting material according to claim 2, wherein R₄ and R₅ are each independently selected from hydrogen and phenyl.

18. The light-emitting material according to claim 1, wherein the structural formula shown in Formula 2 is as follows: [chemical structure]
